(19) United States
(12) Patent Application Publication
(10) Pub. No.: US 2023/0206196 A1
(43) Pub. Date: Jun. 29, 2023

(54) GRANULAR PROCESS FAULT DETECTION

(71) Applicant: Optum Services (Ireland) Limited, Dublin (IE)

(72) Inventors: Lorcan Mac Manus, Maynooth (IE); Peter Cogan, Dublin (IE); David Belton, Dublin (IE); Andrew Kirk, Glenageary (IE)

(21) Appl. No.: 17/646,257

(22) Filed: Dec. 28, 2021

(51) Int. Cl. G06Q 20/10; G06Q 20/16; G06Q 20/38

(52) U.S. Cl. CPC G06Q 20/102; G06Q 20/16; G06Q 20/389

(57) ABSTRACT

A method includes obtaining a series of more than two Boolean events, each respective Boolean event having either a first value indicating that reimbursement for a respective overpayment of a service was timely received or a second value indicating reimbursement was not timely received. The method includes determining an actual number of switch events in the series of Boolean events and determining an expected number of switch events. The method includes determining a confidence interval for the expected number of switch events, and determining a score based on the actual number of switch events, the expected number of switch events, and the confidence interval. The score is indicative of a fault in a process generating the series of Boolean events. The method includes generating an alert based on the score being greater than or equal to a threshold score.

### TECHNICAL FIELD

This disclosure relates to computing systems for fault detection.

### BACKGROUND

A time series data set is a sequence of individual events taken or recorded at successive points in time and which may be equally spaced in time or unequally spaced in time. In other words, a time series data set is a sequence of discrete-time data. Business metrics such as sales, recoveries, and cost savings over time, and the like, are aggregations of sets of underlying time series events (e.g., time series elements) which may or may not be independent. For example, annual sales is the sum of daily customer spend.

Some time series data sets comprise Boolean events/elements, that is, the event/element may be one of two values, e.g., “success”/“failure”, true/false, 1/0, yes/no, and the like. Each event is a Boolean random variable representing a Bernoulli trial, and if the Boolean random variables are independent, the time series data set is the result of a Bernoulli process and have a binomial distribution.

### SUMMARY

The present disclosure describes devices, systems, and methods for determining the presence or absence of a fault in a Boolean time series data set. In some examples, the determination of a fault is based on how well the data set is modeled as a Bernoulli process. The systems and methods disclosed also provide for generating an alert based on a determination of a fault. For example, a fault may comprise a lack of independence between the random variables of the data set and may be exemplified by “stuck behavior,” e.g., where the outcomes of the events/elements do not vary randomly but rather vary based on a special cause, e.g., the fault. In other words, variation in the outcomes of the events/elements of the data set including a fault may not be well-modeled by a Bernoulli process.

The individual events/elements may be considered to be granular “status reports” that may signify and/or indicate the presence or absence of a fault. For example, in a healthcare context, whether a reimbursement of an overpayment to a healthcare service provider is timely received is a Boolean event, e.g., whether or not a reimbursement for an overpayment (e.g., from an insurer or organization) was timely received may have one of two values for each transaction, a “yes” or a “no.” The healthcare service provider may generate “events” such as data and/or information associated with a time series data set that includes of Boolean events, e.g., the amount and dates of transactions such as reimbursements for overpayments of services provided. A time series data set of Boolean events may be derived from the data and/or information generated by the healthcare service provider, e.g., whether reimbursements for overpayments were timely received, and the healthcare service provider may be considered to be an event generator, and alternatively referred to as a Boolean event generator for brevity (although the actual output may not be Boolean events but rather output from which Boolean events may be derived). An overall daily, monthly, annual, and the like, recovery of overpayments depends on the behavior of a plurality (e.g., tens of thousands) of individual healthcare service providers (e.g., Boolean event generators), with each of the plurality of healthcare service providers generating a time series data set of Boolean events, e.g., reimbursements that were timely received or not.

A Bernoulli process is a finite or infinite sequence of independent identically distributed Bernoulli trials, where the outcome of each trial is one of two possibilities (e.g., a first value or a second value, e.g., each Bernoulli trial is a Boolean event) and the probability that a trial is one of the two outcomes (e.g., the first value) is the same for each trial. Summary statistics, such as a maximum likelihood estimate “p”, are readily obtainable from a time series data set of Bernoulli trials/Boolean events, e.g., via calculating the average number of “success” or “true” event outcome values in the data set and the total count of events, “n”, from which a variance may be estimated. Basing determination of a fault on summary statistics, e.g., a higher or lower maximum likelihood estimate, or a maximum likelihood estimate that has changed or drifted, may not be adequate to identify a fault. For example, two healthcare service providers may have the same maximum likelihood estimate p, but one may exhibit random variation in event outcome values and the other may exhibit a fault such as “stuck” behavior (as described with reference to FIG. 3 below). In other words, the time series data sets of reimbursements from some of a plurality of healthcare service providers may be well-modeled as a Bernoulli process. The time series data sets of reimbursements from other providers may not be well-modeled as a Bernoulli process, e.g., having variation in timely reimbursement the may depend on a special cause or fault such as a systemic problem executing transactions, inability to reimburse, a local event causing reimbursement delay (e.g., a natural or manmade disruption to the healthcare service provider's operations), a widespread event causing a disruption to a healthcare service provider's operations (e.g., an epidemic, a pandemic, etc.), or the like. Determination of a fault or special cause, even for providers exhibiting the same maximum likelihood estimate of providing timely reimbursements, is beneficial in determining prioritization of which of the plurality of healthcare service providers should have an escalated analysis, e.g., choosing which of the tens of thousands of providers to spend time and money on further investigating in order to resolve faults.

In one example, this disclosure describes a method including obtaining, by a computing system, a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determining, by the computing system, an actual number of switch events in the series of Boolean events, wherein each switch event in the series of Boolean events is a change from the first value to the second value or the second value to the first value; determining, by the computing system, an expected number of switch events for the series of Boolean events; determining, by the computing system, based on the expected number of switch events and the total number of Boolean events in the series of Boolean events, a confidence interval for the expected number of switch events; determining, by the computing system, a score based on the actual number of switch events, the expected number of switch events, and the confidence interval, wherein the score is indicative of a fault in a process generating the series of Boolean events; and generating, by the computing system, an alert based on the score being greater than or equal to a threshold score.

In another example, this disclosure describes a computing system including a communication unit configured to obtain a plurality of Boolean events; and one or more processors implemented in circuitry and in communication with the communication unit, the one or more processors configured to: obtain a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determine an actual number of switch events in the series of Boolean events, wherein each switch event in the series of Boolean events is a change from the first value to the second value or the second value to the first value; determine an expected number of switch events for the series of Boolean events; determine, based on the expected number of switch events and the total number of Boolean events in the series of Boolean events, a confidence interval for the expected number of switch events; determine a score based on the actual number of switch events, the expected number of switch events, and the confidence interval, wherein the score is indicative of a fault in a process generating the series of Boolean events; and generate an alert based on the score being greater than or equal to a threshold score.

In another example, this disclosure describes a non-transitory computer-readable medium having instructions stored thereon that, when executed, cause one or more processors to: obtain a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determine an actual number of switch events in the series of Boolean events, wherein each switch event in the series of Boolean events is a change from the first value to the second value or the second value to the first value; determine an expected number of switch events for the series of Boolean events; determine, based on the expected number of switch events and the total number of Boolean events in the series of Boolean events, a confidence interval for the expected number of switch events; determine a score based on the actual number of switch events, the expected number of switch events, and the confidence interval, wherein the score is indicative of a fault in a process generating the series of Boolean events; and generate an alert based on the score being greater than or equal to a threshold score. The details of one or more aspects of the disclosure are set forth in the accompanying drawings and the description below. Other features, objects, and advantages of the techniques described in this disclosure will be apparent from the description, drawings, and claims.

The details of one or more examples are set forth in the accompanying drawings and the description below. Other features, objects, and advantages will be apparent from the description, drawings, and claims.

### BRIEF DESCRIPTION OF THE DRAWINGS

FIG. 1 is a block diagram illustrating an example system in accordance with one or more aspects of this disclosure.

FIG. 2 is a block diagram illustrating an example computing system that implements a telemedicine facilitation application in accordance with one or more aspects of this disclosure.

FIG. 3 is a conceptual diagram illustrating example time series data sets in accordance with one or more aspects of this disclosure.

FIG. 4 is a flowchart illustrating an example method of determining a granular process fault in accordance with one or more aspects of this disclosure.

FIG. 5 is a conceptual diagram illustrating example ordered time series data sets in accordance with one or more aspects of this disclosure.

### DETAILED DESCRIPTION

Known and/or conventional approaches to determining a fault (e.g., non-random variation in a data series indicating a special cause, problem, etc., with a process generating the data series) include estimating a summary statistic. One such summary statistic is a maximum likelihood estimate p, e.g., the probability of an outcome of an event of the data series being a particular value. The maximum likelihood estimate p may be determined for a succession of time intervals (constant sampling time) or batch sizes (constant sampling number) for a data series in which events (e.g., the occurrence of a value) occur as a function time. In this way, elements with abrupt changes in p may become apparent. For Boolean events, the “outcome” and/or “value” of each event is binary and may have one of two values, e.g., a “success” or “failure,” “true” or “false,” “1” or “0,” or the like. For Boolean events p+q=1, where p is the probability of a Boolean event outcome being the first value and q is the probability of a Boolean event outcome being the second value.

The known/conventional approaches may be effective for time series data sets having events that occur at a regular rate and or are synchronized between series having different Boolean event generators. For example, a summary statistic such as the maximum likelihood estimate p may be computed for subsets of the data series, e.g., every 10 Boolean events, and a fault may be detected based on a change or rate of change of the summary statistic over time. In this case, for calculating a summary statistic of subsets of the time series data set, the choice of whether to “bin” or accumulate a subset of Boolean events according to a certain number of events or over a regular time period is moot, because the events occur regularly in time. However, the time over which a first Boolean event series generated by a first Boolean event generator and a second Boolean event series generated by a second Boolean event generator occur is unspecified, and each Boolean event comprising the respective series may not have a consistent time between Boolean events. Additionally, the first and second series may not be synchronized or even correlated with each other. In some instances, the first and second series may have the same total number of Boolean events over a particular period of time (e.g., a “time window”), however in other instances, the first and second series may not have the same number of Boolean events over a particular period of time.

Determining a summary statistic via known/conventional approaches with irregular and/or unsynchronized time series data sets presents a choice between how to “bin” subsets of the Boolean events in order to calculate the summary statistic, namely, use a constant time window or a constant number of events window. Calculating a summary statistic using a constant time window allows for straightforward visualization of the summary statistic as a function of time, however, it is difficult to determine what the size of the time window should be. By way of the example described above, a Boolean event generator may be a healthcare service provider, and whether or not the healthcare service provider timely reimbursed an overpayment for a service may be a Boolean event. Some Boolean event generators, such as a large hospital system, may generate 10 times, 100 times, 1000 times, or more, as many reimbursement events as other Boolean event generators, such as a local clinic. As such, there will be a vastly different number of Boolean events for one provider versus another provider leading to different variances in calculated summary statistics and resulting in problematic and inaccurate comparisons between Boolean event generators (e.g., providers). Additionally, inaccuracies may arise when the time window does not align well with a fault in the time series data set. For example, if an edge of a time window applied to a Boolean event series occurs within a run of second values (e.g., failures or no timely reimbursement), the summary statistic of each time window will be averaged and the summary statistic may not indicate a change accurately reflecting the magnitude of the fault since its underlying Boolean event data is split between the two windows.

As to the other choice, calculating a summary statistic using a constant number of events window allows summary statistics with similar variances to be calculated, however, the summary statistics between different Boolean event generators (providers) will now be asynchronous with time making comparisons across time problematic and/or impossible. Additionally, the problem of choosing the correct window size still remains, as does the problem with the time window not aligning well with a fault.

Known and/or conventional approaches to determining a fault, e.g., based on a summary statistic, are time consuming and require extensive computing resources and computing power. For example, known/conventional approaches require extra computing power (e.g., requiring more time and/or more physical computing resources, cost, and electrical power) to compensate for a plurality of Boolean event series having different total numbers of Boolean events that are not aligned in time, e.g., generated at different rates by the different Boolean event generators. For example, known and/or conventional approaches may calculate additional metrics beyond summary statistics, and/or may implement increasingly complex algorithms to compensate for aligning Boolean events in time and/or to compensate for differing variances in order to arrive at improved comparisons between Boolean event generators.

In accordance with one or more techniques of this disclosure, a fault may be determined by how well a first order difference series, or “switching series” is modeled as a Bernoulli processes. A first order difference series is a series corresponding to a Boolean event series in which each element of the first order difference series is the difference between two adjacent (e.g., in time) Boolean events in the Boolean event series, e.g., a “no switch” if two consecutive Boolean events are the same and a “switch” if the Boolean events are different. The probability of switching between values of a Bernoulli process is itself a Bernoulli process having its own confidence intervals, and a first order difference series is itself a Boolean event series. In other words, a first order difference series may be well-modeled or poorly-modeled as Bernoulli processes, e.g., based on how well its respective corresponding Boolean event series is modeled as Bernoulli processes. If a first order difference series, or its corresponding Boolean event series, is not well-modeled as a Bernoulli process, it is likely there is a special cause and/or fault causing the series to behave differently than a Bernoulli process. The techniques of this disclosure provide improved fault detection and/or fault determination requiring less computing power and/or computing resources.

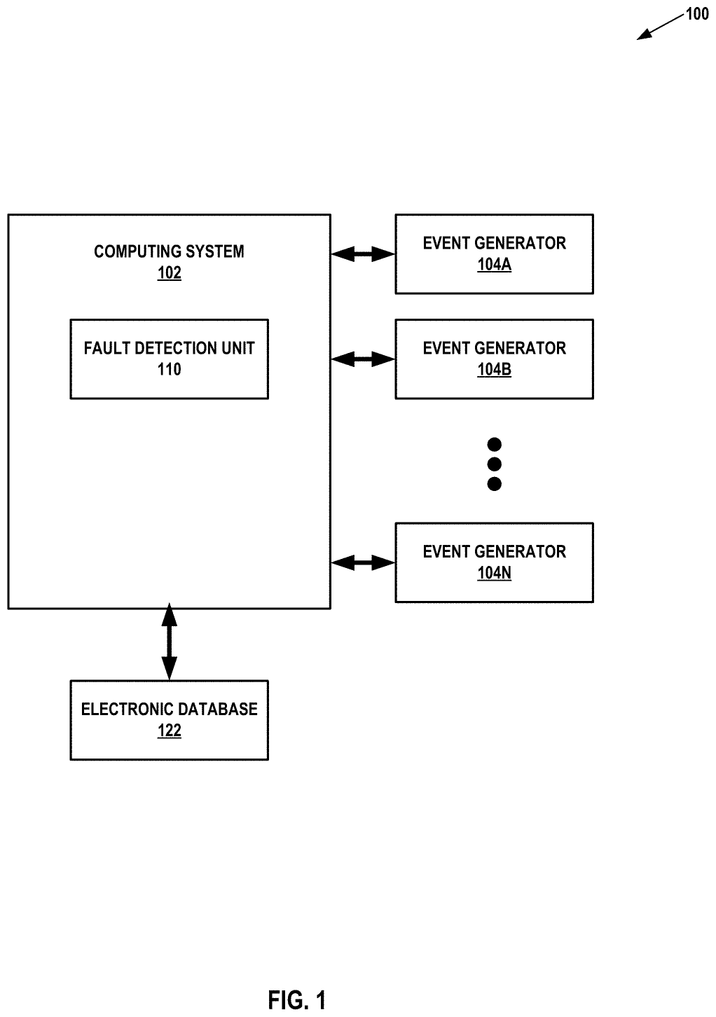

FIG. 1 is a block diagram illustrating an example system 100 in accordance with one or more aspects of this disclosure. In the example of FIG. 1, system 100 includes a computing system 102, fault detection unit 110, an electronic database 122, and a plurality of event generators 104A, 104B, and 104N, which are collectively referred to herein as “event generators 104.”

Fault detection unit 110 may operate on computing system 102. In some examples, computing system 102 may include one or more computing devices. In examples where computing system 102 includes two or more computing devices, the computing devices of computing system 102 may act together as a system and may be distributed. Example types of computing devices include server devices, personal computers, mobile devices (e.g., smartphones, tablet computers, wearable devices), intermediate network devices, and so on.

In the example shown, event generators 104 output data and/or information associated with a time series data set including a plurality of Boolean events, as mentioned above. The Boolean events may or may not be independent and may or may not be random. In the example shown, event generators 104 may be healthcare service providers that may output information and/or data (e.g., the amount and dates of transactions such as reimbursements for overpayments of services provided) associated with Boolean events (e.g., whether a reimbursement was timely received), however, in other examples, event generators 104 may be any other entity that may output a time series data set including a plurality of Boolean events or data and/or information associated with a time series data set of Boolean events.

For example, a healthcare service provider may provide healthcare services and receive a payment for the services provided from a healthcare insurer or other organization. The payment received may be an overpayment, for which healthcare service provider may be required to reimburse the insurer and/or organization within a period of time, e.g., within 30 days, 60 days, 90 days, or any other suitable or agreed upon time period. Whether a reimbursement is received within the time period (timely received) or not is a Boolean event, and the Boolean event may be received by computing system 102 and recorded and/or stored in electronic database 122. For example, if the healthcare service provider timely reimburses for an overpayment, the Boolean event is a “success” (or a “True” or “1” or any other suitable way of denoting a first value of the Boolean event outcome). If the healthcare service provider does not timely reimburse for the overpayment, the Boolean event is a “failure” (or “False” or “0” or any other suitable way of denoting a second value of the Boolean event). A healthcare service provider may provide a plurality of services over time for which the provider receives an overpayment and is required to timely reimburse, and whether the provider timely reimbursed the overpayments is a time series data set of Boolean events.

In some examples, Boolean event values/outcomes may be stored in electronic database 122 (e.g., True/False, 1/0, Yes/No). In some examples, information and/or data from which Boolean event values/outcomes may be derived is stored in electronic database 122 For example, transaction details may be stored in electronic database 122 from which a Boolean event value/outcome may be derived, e.g., transaction details such as amounts, times, dates, and identifiers linking the transactions to a service provided may be stored in electronic database 122 from which fault detection unit 110 may determine the outcome of the Boolean event, e.g., whether a payment for a service was an overpayment and whether reimbursement of the overpayment was timely received.

In accordance with one or more techniques of this disclosure, fault detection unit 110 may be configured to obtain a time series data set of Boolean events. For example, fault detection unit 110 may be configured to receive data from electronic database 122 containing one or more Boolean events, or data and/or information from which Boolean events may be determined (e.g., transaction data stored in electronic database 122). In some examples, fault detection unit 110 may be configured to obtain a series of Boolean events including more than two Boolean events, with each respective Boolean event in the series having either a first value (e.g., “success”) or a second value (e.g., “failure”). In some examples, the first value indicates reimbursement for overpayment of a service was timely received and the second value indicating that reimbursement for the respective overpayment of the service was not timely received. In other examples, the first and second values may indicate any suitable binary values. In some examples, the service is a healthcare service provided by a healthcare service provider.

Fault detection unit 110 may be configured to determine an actual number of switch events in the obtained series of Boolean events. For example, fault detection unit 110 may be configured to obtain a series of Boolean events corresponding to provider A over a period of time, and a switch event may be a change from the first value to the second value between successive events in the time series, or a switch event may be a change from the second value to the first value between successive events in the times series. For example, if fault detection unit 110 obtains a series of three Boolean events comprising success, failure, success, fault detection unit 110 may determine the actual number of switch events to be two switch events.

Fault detection unit 110 may be configured to determine an expected number of switch events for the series of Boolean events. For example, fault detection unit 110 may be configured to determine the expected number of switch events based on the proportion of success events in the series, the probability of switching if the previous event value is “success”, the proportion of failure events in the series, and the probability of switching if the previous event value is “failure.” Fault detection unit 110 may be configured to determine a confidence interval for a Bernoulli process based on the expected number of switch events and the total number of Boolean events in the series of Boolean events. Fault detection unit 110 may be configured to determine a score based on the actual number of switch events, the expected number of switch events, and the confidence interval. For example, the score may characterize how well the series of Boolean events is modeled by the Bernoulli process. Fault detection unit 110 may be configured to cause computing system 102 to generate an alert based on the score. For example, fault detection unit 110 may cause computing system 102 to generate an alert based on the score being greater than, less than, or equal to a threshold score.

For example, fault detection unit 110 may be configured to determine a score that may characterize how well a series of whether a healthcare service provider timely reimbursed overpayments is modeled by a Bernoulli process. If the score is high, indicating that the series is not well-modeled as a Bernoulli process, fault detection unit 110 may output an alert if the score is greater than a threshold score, indicating that there is an increased likelihood that there is a special cause and/or fault interfering with timely reimbursements from the healthcare service provider. In some examples, the score may be comparable across a plurality of healthcare service providers each generating events (timely reimbursement of an overpayment or not) at the same or different rates, and may be used to prioritize which healthcare service providers to further investigate.

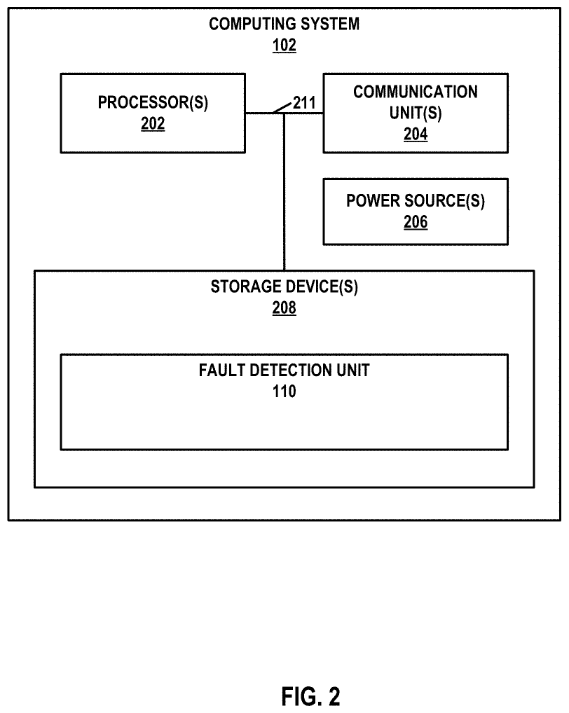

FIG. 2 is a block diagram illustrating example components of computing system 102 in accordance with one or more aspects of this disclosure. FIG. 2 illustrates only one example of computing system 102, without limitation on many other example configurations of computing system 102, and may comprise a separate system of one or more computing devices.

As shown in the example of FIG. 2, computing system 102 includes one or more processors 202, one or more communication units 204, one or more power sources 206, one or more storage devices 208, and one or more communication channels 211. Computing system 102 may include other components. For example, computing system 102 may include input devices, output devices, display screens, and so on. Communication channel(s) 210 may interconnect each of processor(s) 202, communication unit(s) 204, and storage device(s) 208 for inter-component communications (physically, communicatively, and/or operatively). In some examples, communication channel(s) 210 may include a system bus, a network connection, an inter-process communication data structure, or any other method for communicating data. Power source(s) 206 may provide electrical energy to processor(s) 202, communication unit(s) 204, storage device(s) 206 and communication channel(s) 210. Storage device(s) 208 may store information required for use during operation of computing system 108.

Processor(s) 202 comprise circuitry configured to perform processing functions. For instance, one or more of processor(s) 202 may be a microprocessor, an application-specific integrated circuit (ASIC), a field-programmable gate array (FPGA), or another type of processing circuitry. In some examples, processor(s) 202 of computing system 102 may read and execute instructions stored by storage device(s) 208. Processor(s) 202 may include fixed-function processors and/or programmable processors. Processor(s) 202 may be included in a single device or distributed among multiple devices.

Communication unit(s) 204 may enable computing system 102 to send data to and receive data from one or more other computing devices (e.g., via a communications network, such as a local area network or the Internet). In some examples, communication unit(s) 204 may include wireless transmitters and receivers that enable computing system 102 to communicate wirelessly with other computing devices. Examples of communication unit(s) 204 may include network interface cards, Ethernet cards, optical transceivers, radio frequency transceivers, or other types of devices that are able to send and receive information. Other examples of such communication units may include BLUETOOTH™, 3G, 4G, 5G, and WI-FI™ radios, Universal Serial Bus (USB) interfaces, etc. Computing system 102 may use communication unit(s) 204 to communicate with one or more other computing devices or systems, such as electronic database 122. Communication unit(s) 204 may be included in a single device or distributed among multiple devices.

Processor(s) 202 may read instructions from storage device(s) 208 and may execute instructions stored by storage device(s) 208. Execution of the instructions by processor(s) 202 may configure or cause computing system 102 to provide at least some of the functionality ascribed in this disclosure to computing system 102. Storage device(s) 208 may be included in a single device or distributed among multiple devices.

As shown in the example of FIG. 2, storage device(s) 208 may include computer-readable instructions associated with fault detection unit 110. Fault detection unit 110 shown in the example of FIG. 2 is presented for purposes of explanation and may not necessarily correspond to actual software units or modules within storage device 208.

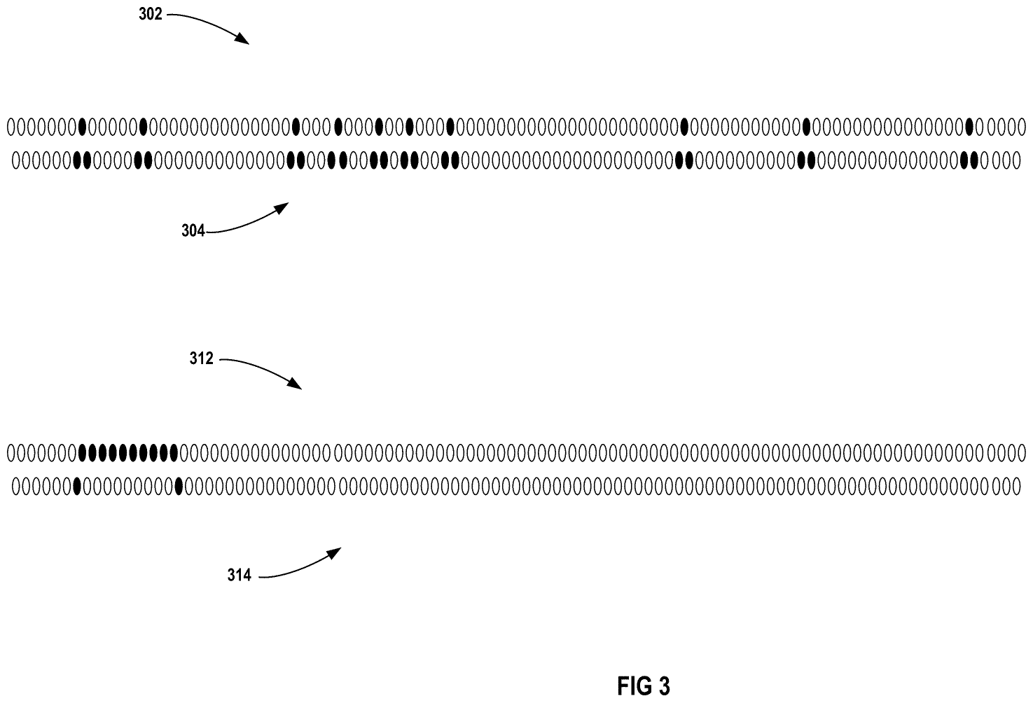

As described above, fault detection unit 110 is configured to obtain data indicating a series of Boolean events and determine score characterizing how well the series of Boolean events is modeled by a Bernoulli process, and to generate an alert if that score is at or below, (or at or above) a threshold score. The operation of fault detection unit 110 is described with reference to the example Boolean time series data sets 302 and 312 of FIG. 3.

FIG. 3 is a conceptual diagram illustrating example time series data sets in accordance with one or more aspects of this disclosure. In the example shown in FIG. 3, Boolean time series data set 302 (e.g., Boolean event series 302) corresponds to 100 Boolean events (e.g., N=100) generated by Boolean event generator 104A, e.g., 100 reimbursement Boolean events (referred to as “series 302” or “provider A series 302” for brevity). Boolean event series 312 corresponds to 100 Boolean events generated by Boolean event generator 104B, e.g., 100 reimbursement Boolean events.

In the examples of FIG. 3, both provider A series 302 and provider B series 312 include 90 “successful” events denoted by the 90 unfilled ovals of series 302 (e.g., timely reimbursement) and 10 “failure” events denoted by the 10 filled ovals of series 302. The nominal probability of a “failure” of both series 302 and series 312 is the same, e.g., 10%, however, the “failure” events of series 302 are statistically randomly distributed throughout the series and the “failure” events of series 312 are clumped together in a single group. As such, there is a high probability that provider A series 302 is well-modeled as a Bernoulli process, and provider B series 312 is not well-modeled as a Bernoulli process as exemplified by the “stuck” behavior of the clumped Boolean events. In other words, the “failure” events of provider A series 302 occur randomly throughout the series of events, but the “failure” events of provider B series 312 occur consecutively with a very low probability of having occurred by chance and a high probability of being caused by a fault or special cause.

In the example shown in FIG. 3, series 304 and series 314 are switching series corresponding to Boolean event series 302 and 312, respectively. For example, each event of series 304 corresponds to whether a subsequent event of series 302 switches from the first value to the second value or from the second value to the first value. For example, for the first seven events of series 302, each event is the first value, e.g., a “success” denoted by seven unfilled ovals. Therefore, the first six elements of corresponding series 304 are a first switch value, e.g., “no switch” denoted by six unfilled ovals of series 304. The eighth event of series 302 is the second value, e.g., “failure” indicating no timely reimbursement received and denoted by a filled oval, and is a switch in value between successive Boolean events in time. Therefore the seventh element of series 304 is the second value, e.g., “yes switch” denoted by a filled oval. The ninth Boolean event of series 302 is the first value, e.g., another switch between values of successive Boolean events in time, and as such the eighth element of series 304 is again the second value as denoted by a filled oval. In the example shown, because Boolean event series 302 contain no successive “failure” values, switching series 304 includes 20 “yes switch” values denoting 20 switches between Boolean values for series 302. By way of contrast, Boolean event series 312 contains only successive “failure” values, and switching series 314 includes only 2 “yes switch” values denoting 2 switches between Boolean values for series 312. Although the examples of FIG. 3 of switching series 304 and 314 include “yes switch” elements for each switch from either the first value to the second and vice versa of the respective Boolean event series 302 and 312, in other examples switching series 304 and 314 may include only “yes switch” elements for a change from the first to the second value of the respective series 302 and 312, or only “yes switch” elements for a change from the second to the first value of the respective series 302, and 312.

In contrast to known/conventional techniques, e.g., described above, the techniques described herein do not require calculating a summary statistic for analysis and/or comparison based on subsets of a time series data set. In accordance with one or more techniques of this disclosure, fault detection unit 110 may be configured to determine how well switching series 304 and 314 are modeled as Bernoulli processes. The probability of switching between values of a Bernoulli process is itself a Bernoulli process having its own confidence intervals. In other words, switching series 304 and 314 may be well-modeled or poorly-modeled as Bernoulli processes, e.g., based on how well their respective corresponding Boolean event series 302 and 312 are modeled as Bernoulli processes.

A time series data set of Boolean events that is well-modeled as a Bernoulli process has p proportion of individual Boolean event outcomes as the first value (success) and 1−p proportion of outcomes as the second value (failure). If the current Boolean event outcome is the second value, the probability of switching on the next successive Boolean event is p. If the current Boolean event outcome is the first value, the probability of switching on the next successive Boolean event is 1−p. For Bernoulli process, the expected number of switches is the product of the proportion of events at the first value and the probability of switching if at the first value plus the product of the proportion of events at the second value and the probability of switching if at the second value. In equation form, the expected switch probability (e.g., or rate) is p_{sw}=2*p*(1−p), and the expected number of switches is N_{sw}=p_{sw}*N=2*p*(1−p)*N, where N is the total number of Boolean events in the data set. The confidence interval for such a Bernoulli process is the

$$CI = \sqrt{\frac{{p}_{sw} * ( 1 - {p}_{sw} )}{N}} .$$

The actual number of switches, e.g., of switch series 304 and/or 314, may be determined via counting the “yes switch” elements. In some examples, switch series 304 and 314 may be referred to as first order differences of series 302 and series 312, respectively. For example, it is often convenient to represent the first and second values of series 302 and 312 as 1's (successes) and 0's (failures). A first order difference series is then the result of the difference between each successive Boolean event value, e.g., n₂−n₁, n₂−n₁, etc., where nₓ is the x^{th} Boolean event in the series. The first order difference series (e.g., switch series 304 and switch series 314) may then comprise 1's (a switch from the second value to the first value), 0's (no switch), and −1's (a switch from the first value to the second value). The actual number of switches may then be calculated as the sum of the absolute values of all of the elements of the first order difference series. An estimated switch probability, p′, may be based on the actual number of switches, e.g., the actual number of switches divided by the total number of elements in the first order difference series. For example, p′=N_{sw-actual}|(N−1), where N_{sw-actual} is the actual number of switches and N is the total number of Boolean events (e.g., the total number of elements in the first order difference series is the total number of possible switches, and is one less than the total number Boolean events).

In the examples shown, each of Boolean event series 302 and series 312 have a p=0.9 proportion of the first value (e.g., 90 unfilled ovals indicating “successes” or “1's”). The expected switch probability for each series is p_{sw}=0.18, with a confidence interval CI=0.038. In other words, the expected number of switches for each series is 18 (e.g., the total number N*p_{sw}). A Boolean event series having a first order difference series with between 14 and 22 switches (e.g., N*(p_{sw}+/−CI) may be considered to be well-modeled as a Bernoulli process and variation in the series is very likely random rather than special cause. Switching series 304 corresponding to provider A series 302 has 20 switches, and may be considered to be well-modeled as a Bernoulli process and likely does not include a fault. Switching series 314 corresponding to provider B series 304 has 2 switches, and may be considered to be not well-modeled as a Bernoulli process and variation in provider B series 304 is likely not random variation but rather due to a special cause, e.g., a fault.

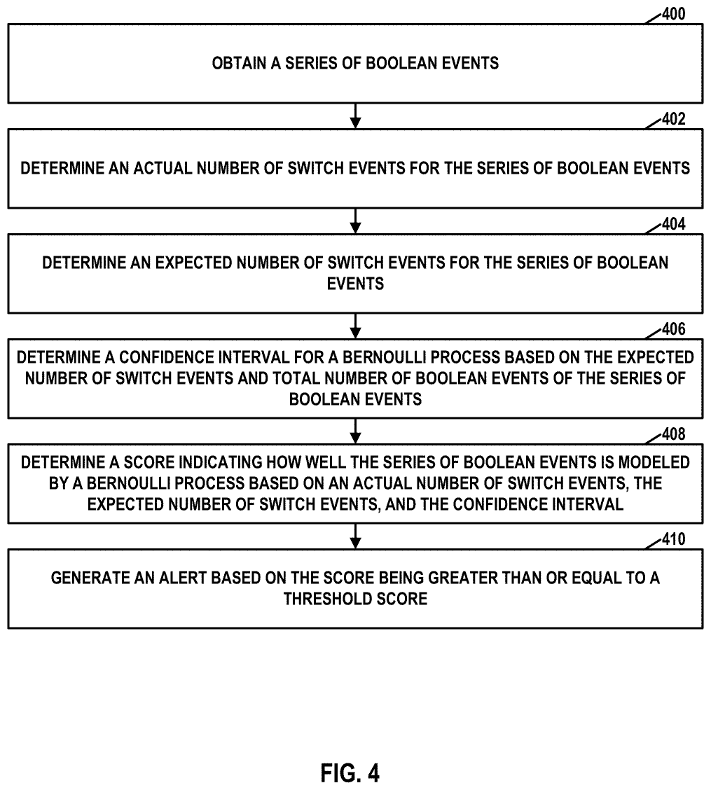

FIG. 4 is a flowchart illustrating an example method of determining a score indicative of a fault in a process generating Boolean events in accordance with one or more aspects of this disclosure. In some examples, the method may be an example of the operation of fault detection unit 110. In other examples, the method and/or operations of fault detection unit 110 may include more, fewer, or different actions. The flowchart of this disclosure is described with respect to the other figures of this disclosure. However, the flowchart of this disclosure is not so limited. For ease of explanation, the flowchart of this disclosure is described with respect to system 100 and computing system 102, but the flowchart of this disclosure may be applicable mutatis mutandis with respect to privacy aspects of system 100.

In the example of FIG. 4, fault detection unit 110 may obtain a series of Boolean events (400). For example, fault detection unit 110 may receive data from electronic database 122 containing one or more Boolean events, or data from which Boolean events may be determined (e.g., transaction data stored in electronic database 122). In some examples, fault detection unit 110 may obtain a series of Boolean events including more than two Boolean events, with each respective Boolean event in the series having either a first value (e.g., “success”) or a second value (e.g., “failure”). In some examples, the first value indicates reimbursement for overpayment of a service was timely received and the second value indicating that reimbursement for the respective overpayment of the service was not timely received. In other examples, the first and second values may indicate any suitable binary values. In some examples, fault detection unit 110 may obtain a series of Boolean events that are ordered in time.

Furthermore, in the example of FIG. 4, fault detection unit 110 may determine an actual number of switch events in the obtained series of Boolean events. For example, fault detection unit 110 may obtain a series of Boolean events corresponding to provider A over a period of time, and a switch event may be a change from the first value to the second value between successive events in the time series, or a switch event may be a change from the second value to the first value between successive events in the times series. For example, if fault detection unit 110 obtains Boolean event series 302, fault detection unit 110 may determine the actual number of switch events of Boolean event series 302 to be 20 switch events. In some examples, fault detection unit 110 may determine a first order difference of the obtained Boolean event series. For example, fault detection unit 110 may determine switch series 304, e.g., the elements of which are the first order differences of Boolean event series 302. In some examples, fault detection unit 110 may determine the actual number of switch events based on one or both of Boolean event series 302 and switch series 304.

Fault detection unit 110 may determine an expected number of switch events for the series of Boolean events (404). For example, fault detection unit 110 may determine a proportion of outcomes of the Boolean events of Boolean event series 302 being the first value as 0.9, e.g., 90 out of 100 unfilled ovals indicating “success” or “1” values. The proportion of outcomes of the total N=100 Boolean events of Boolean event series 302 may be an estimate of the probability p=0.9 of an outcome of a Boolean event generated by an underlying process, e.g., the Boolean event generator being provider A's process of reimbursing overpayments in the example of Boolean event series 302. Fault detection unit 110 may determine the expected number of switch events as N_{sw}=p_{sw}*N=2*p*(1−p)*N, as described above.

Fault detection unit 110 may determine a confidence interval for the expected number of switch events based on the expected number of switch events and the total number of Boolean events in the series of Boolean events (406). For example, fault detection unit 110 may determine a confidence interval

$$CI = \sqrt{\frac{{p}_{sw} * ( 1 - {p}_{sw} )}{N}} ,$$

as described above.

In some examples, fault detection unit 110 may determine the expected number of switch events for a Boolean event series based on an Agresti-Coull method. An Agresti-Coull method may estimate a confidence interval via an Agresti-Coull interval in which a number of first and second values are added to the Boolean event series, e.g., at least one first value and at least one second value. In some examples, two of the first value (e.g., successes) and two of the second value (e.g., failures) are added to the Boolean event series (although any number of first and second values may be added in other examples). An Agresti-Coull method may improve the estimation of the confidence interval by avoiding spurious results when N is small. For example, fault detection unit 110 may determine the estimated probability of an outcome of a Boolean event of series 302 being the first value by adding 2 “successes” to series 302. Fault detection unit 110 may also add 2 “failures” to series 302, increasing N to 104. As such, fault detection unit 110 may determine the estimated first value probability as p1=(n1+2)/(N+4), where n1 is the original first value count in the series (e.g., 90 for series 302). Fault detection unit 110 may then determine the expected switch probability using p1 rather than p, namely, p_{sw}=2*p1*(1−p1). Fault detection unit 110 may then determine the confidence interval CI a described above.

Fault detection unit 110 may determine a score indicative of a fault in a process generating the series of Boolean events based on the actual number of switch events, the expected number of switch events, and the confidence interval (408). For example, fault detection unit 110 may determine the score as the absolute value of the difference between the expected switch probability and the estimated switch probability, p, divided by the switch confidence interval CI, e.g.,

$$score = \frac{\vert {p}_{sw} - p ′ \vert}{CI} .$$

Fault detection unit may determine the estimated switch probability p′ as described above at FIG. 3, e.g., p′=N_{sw-actual}| (N−1).

In some examples, the score may be a t-score or a t-statistic. In some examples, fault detection unit 110 may determine the score based on an Agresti-Coull method. For example, fault detection unit 110 may determine the score as the absolute value of the difference between the expected switch probability calculated using an Agresti-Coull method as described above (e.g., p_{sw}=2*p1*(1−p1)) and an estimated switch probability p1′ based on an Agresti-Coull method divided by the switch confidence interval CI, e.g.,

$$score = \frac{\vert {p}_{sw} - p 1 ′ \vert}{CI} .$$

Fault detection unit 110 may determine the estimated switch probability p1′ based on an Agresti-Coull method by adding two switch events and two non-switch events to the first order difference series, namely, p1′=(N_{sw-actual}+2)/[(N−1)+4], where N_{sw-actual} is the actual number of switches and N is the total number of Boolean events.

By way of specific example, fault detection unit 110 may determine a first score for a first series of Boolean events, e.g., Boolean event series 302 of FIG. 3, and a second score for a second series of Boolean events, e.g., Boolean event series 312 of FIG. 3. For example, fault detection unit 110 may obtain both Boolean event series 302 and 312, e.g., from electronic database 122, and determine a first number of actual switch events e.g., N_{sw-actual}=20 for the first series of Boolean events and a second number of actual switch events N_{sw-actual}=2 for the second series of Boolean events, as described above at FIG. 3. Fault detection unit 110 may determine a first expected number of switch events as 18, e.g., N_{sw}=p_{sw}*N=2*p*(1−p)*N, where p is the maximum likelihood estimate (e.g., 90/100=0.9 for Boolean event series 302) and N is the total number of Boolean events (e.g., N=100 for Boolean event series 302). Fault detection unit 110 may determine a second expected number of switch events in the same way for the second Boolean event series. For Boolean event series 302 and 312, fault detection unit 110 determine the same N_{sw}=18, since each series has the same number of Boolean events having the first values and the same total number of Boolean events. Fault detection unit 110 may then determine first and second confidence intervals, e.g.

$$CI = \sqrt{\frac{{p}_{sw} * ( 1 - {p}_{sw} )}{N}} ,$$

which may be the same for Boolean event series 302 and 312, e.g., CI=0.038. In some examples, fault detection unit 110 may determine the first and second confidence intervals, based on the first and second numbers of expected switch events, e.g., N_{sw}=p_{sw}*N, and the first and second total number of switch events, e.g., N, namely

$$CI = \sqrt{\frac{\frac{{N}_{sw}}{N} * ( 1 - \frac{{N}_{sw}}{N} )}{N}} .$$

Fault detection unit 110 may then determine the first score based on the first actual number of switch events, e.g., N_{sw-actual}=(N−1)*p′ as described above at FIG. 3, the first expected number of switch events N_{sw}, and the first confident interval CI, namely,

$$the first score = \frac{\vert {p}_{sw} - p ′ \vert}{CI} = \frac{\vert \frac{{N}_{sw}}{N} - \frac{{N}_{sw - actual}}{( N - 1 )} \vert}{CI} = 0.57 .$$

Fault detection unit 110 may determine the second score in the same way, e.g., the second score=4.16.

In some examples, fault detection unit 110 may determine the first and second scores according to an Agresti-Coull method. By way of the example above with Boolean event series 302 and 312, two first values and two second values are added to each series, with four added to the total number of Boolean events of each series, and two actual switches added to the first order difference series and four added to the total number of elements of the first order difference series. Fault detection unit 110 may then determine the first actual number of switches to be 22, the second actual number of switches to be 4, the first and second expected number of switches to be 21, and the first and second confidence intervals to be 0.0395. Fault detection unit 110 may then determine the first score to be 0.187 and the second score to be 4.192.

Fault detection unit 110 may generate an alert based on the score being greater than or equal to a threshold score (410). For example, fault detection unit 110 may cause computing system 102 to emit a sound, flash a light, or display an indicator such as within a graphical user interface of a display of computing system 102, based on the score being greater than or equal to a threshold score. In some examples, the threshold score may be about 1.64 (a 95% level), or about 2.33 (a 99% level). For example, a score equal to or above 1.64 may indicate that the process generating the Boolean event series may have a fault causing that process to output Boolean event outcomes that vary based on the fault rather than random variation with a 95% statistical certainty, or a score equal to or above 2.33 may similarly indicate a fault with a 99% statistical certainty.

In some examples, fault detection unit 110 may determine a score indicative of a fault of a Boolean event generator for a plurality of Boolean event generators. For example, fault detection unit 110 may be configured to repeat (400)-(410) for a second Boolean event generator (e.g., provider), such as provider B and Boolean event series 312. Fault detection unit 110 may determine a score indicative of a fault as described above including method steps (400)-(410) for a plurality of Boolean event generators

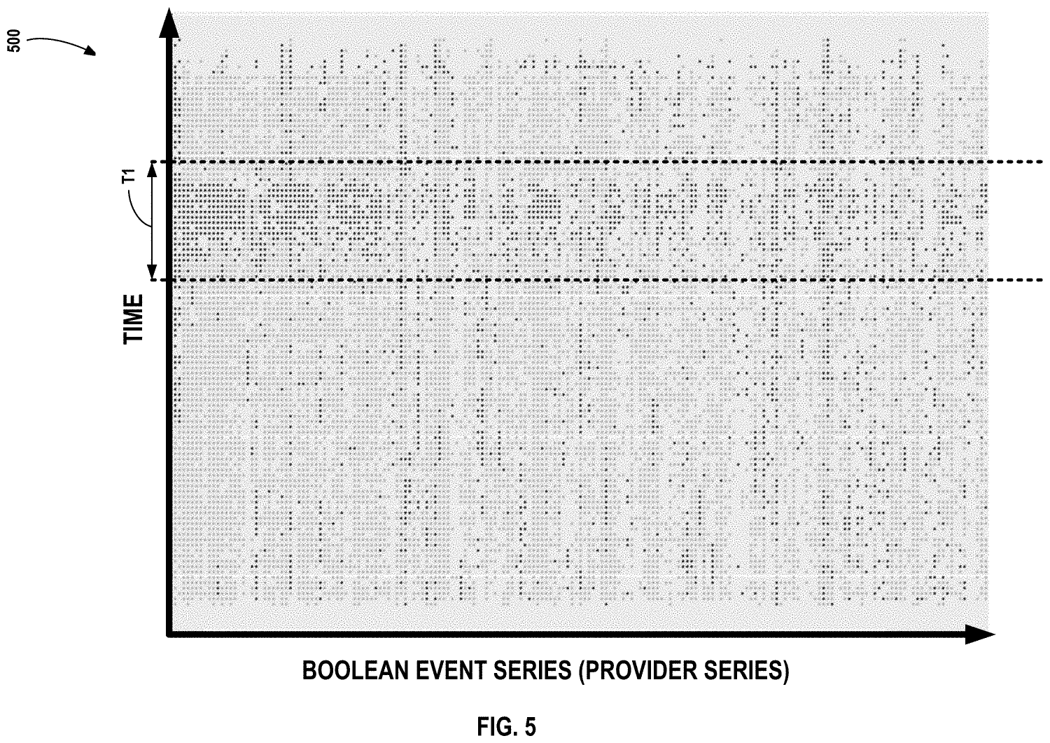

FIG. 5 is a conceptual diagram illustrating example ordered time series data sets 500 in accordance with one or more aspects of this disclosure. In the example shown, a plurality of Boolean event series generated by a plurality of Boolean event generators (e.g., healthcare service providers) are ordered according to score from highest (at left near the time axis) to lowest. Each series includes a plurality of Boolean events along the time axis, e.g., as a column of light and dark dots. The outcomes/values of each Boolean event in the series are indicated by light or dark dots. The light dots indicate a first value such as a “success” and the dark dots indicate a second value, such as a “failure.” In the example shown, several of the plurality of Boolean event series do not include light or dark dots for a period of time, indicating that no Boolean events occurred during that time period for that Boolean event series, e.g., no reimbursement events for that particular provider occurred during the time period.

In some examples, fault detection unit 110 may be configured to determine a metric indicative of a systemic fault affecting a plurality of Boolean event generators, e.g., healthcare service providers in the example shown. For example, time period T1 of ordered time series data sets 500 includes significantly more Boolean events having the second value, e.g., timely reimbursement “failures” for almost all of the providers shown. In the example, time T1 may correspond to an initial period of a pandemic affecting a signification portion of the entire healthcare industry and causing at least temporary disruptions in billing and payment systems, both by healthcare service providers and by health insurance companies and organizations. Although the systemic fault of time period T1 is easily detected visually in the ordered time series data sets 500, fault detection unit 110 may be configured to detect and/or determine systemic faults that are more subtle and may not be visually detectable via one or more methods of presenting and/or displaying the Boolean event series values/outcomes.

For example, fault detection unit 110 may be configured to determine a first number of second values or “failures” and a first total number of Boolean events for a first predetermined time window for a plurality of providers (e.g., each provider associated with a Boolean event series). The first predetermined time window may be a portion of a total time period. For example, the first time window may be an hour, a day, a week, a month, and the like, with a one year total time period (or any other suitable total time period greater than the portion of the total time period). Fault detection unit 110 may determine a first number of “failures” F and a first total number of Boolean events across all providers N, e.g., from electronic database 122, or a subset of all providers, within the first time window. In some examples, fault detection unit 110 may be configured to determine a population failure rate (e.g., a population second value rate) π₀ for the population of Boolean events for the entire total time period and across the plurality of providers (e.g., Boolean event generators). Fault detection unit 110 may be configured to repeat the process for a second time window and/or all of a plurality of time windows within the total time period. For example, fault detection unit 110 may be configured to determine a second total number of second values, a second total number of Boolean events for a second predetermined time window for the plurality of providers. In some examples, the null hypothesis is that each time interval (e.g., time window) has the same population failure, e.g., second value rate. A failure/second value rate of a particular time window that is significantly different than the population failure rate π₀ may be indicative of a systemic fault across a plurality of providers within that time window. In some examples, the plurality of time windows may have the same time interval, be non-overlapping, and/or be adjacent in time (e.g., no time gap between). In other examples, the plurality of time windows may have different time intervals, may overlap, and/or may be not adjacent in time, e.g., may have time gap between two consecutive time windows.

Fault detection unit 110 may be configured to determine a statistical p-value, for a plurality of time windows within the total time period based on N, F, and π₀. For example, fault detection unit 110 may be configured to determine a p-value for each of the plurality of time windows (e.g., a first p-value for the first time window, a second p-value for the second time window, and the like) based on equation 1:

$$p = {\sum }_{k = F}^{N} ( \begin{matrix}N \\ k\end{matrix} ) {{π}_{0}^{k} ( 1 - {π}_{0} )}^{N - k} \qquad ( 1 )$$

Fault detection unit 110 may be configured to order and/or rank each time window by p-value, and may be configured to determine which time windows have a failure rate that is statistically significantly greater than the population average failure rate, e.g., and to determine that these are the time windows with an increased likelihood of having a systemic fault. In some examples, fault detection unit 110 may be configured to determine whether each of the time windows includes a systemic fault based on each time window having a p-value that is less than or equal to a threshold p-value, e.g., whether the first time window has a p-value less than or equal to the threshold p-value, whether the second time window has a p-value less than or equal to the threshold p-value, and the like.

In some examples, fault detection unit 110 may be configured to determine multiple p-values and multiple p-value orderings/rankings. For example, fault detection unit 110 may be configured to repeat the process described above for a varying time window size and number of time windows within the total time period. In some examples, fault detection unit 110 may be configured to determine p-values and a p-value ranking for a first time window (e.g., daily), a second time window (e.g., weekly), and a third time window (e.g., monthly). In some examples, fault detection unit 110 may be configured to use more or fewer than three time window sizes. In some examples, detection of a systemic fault may depend on the size of the time window, and fault detection unit 110 may be configured to search for a suitable and/or optimal time window to detect a systemic fault for the total time period and plurality of Boolean event generators/providers.

The following is a non-limiting list of examples that are in accordance with one or more techniques of this disclosure.

Example 1: A method comprising: obtaining, by a computing system, a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determining, by the computing system, an actual number of switch events in the series of Boolean events, wherein each switch event in the series of Boolean events is a change from the first value to the second value or the second value to the first value; determining, by the computing system, an expected number of switch events for the series of Boolean events; determining, by the computing system, based on the expected number of switch events and the total number of Boolean events in the series of Boolean events, a confidence interval for the expected number of switch events; determining, by the computing system, a score based on the actual number of switch events, the expected number of switch events, and the confidence interval, wherein the score is indicative of a fault in a process generating the series of Boolean events; and generating, by the computing system, an alert based on the score being greater than or equal to a threshold score.

Example 2: The method of example 1, wherein determining the expected number of switch events is based on a total number of Boolean events in the series of Boolean events being the first value and the total number of Boolean events.

Example 3: The method of example 1 or example 2, wherein the score is determined based on an Agresti-Coull method.

Example 4: The method of any one of examples 1 through 3, wherein the series of Boolean events are ordered in time.

Example 5: The method of any one of examples 1 through 4, wherein the service is a healthcare service, wherein the service is provided by a healthcare service provider.

Example 6: The method of any one of examples 1 through 5, wherein the series of Boolean events is a first series of Boolean events, wherein the total number of Boolean events is a first total number of Boolean events, wherein service is a first service, wherein the actual number of switch events is a first actual number of switch events, wherein the expected number of switch events is a first expected number of switch events, wherein the confidence interval is a first confidence interval, wherein the score is a first score, the method further includes obtaining, by a computing system, a second series of Boolean events, wherein: a second total number of Boolean events in the second series of Boolean events is greater than two, and each respective Boolean event in the second series of Boolean events has either the first value or the second value, the first value indicating that reimbursement for a respective overpayment of a second service was timely received, the second value indicating that reimbursement for the respective overpayment of the second service was not timely received; determining, by the computing system, a second actual number of switch events in the second series of Boolean events, wherein each switch event in the second series of Boolean events is a change from the first value to the second value or the second value to the first value; determining, by the computing system, a second expected number of switch events for the second series of Boolean events; determining, by the computing system, based on the second expected number of switch events and the second total number of Boolean events in the series of Boolean events, a second confidence interval for a Bernoulli process; determining, by the computing system, a second score based on the second actual number of switch events, the second expected number of switch events, and the second confidence interval, wherein the second score characterizes how well the second series of Boolean events is modeled by the Bernoulli process; and generating, by the computing system, an alert based on the second score being greater than or equal to a threshold score.

Example 7: The method of example 6, further comprising: determining, by the computing system, a metric indicative of a systemic fault affecting a plurality of Boolean event series within a time period.

Example 8: The method of example 7, wherein determining the metric indicative of a systemic fault comprises: determining, by the computing system, a first total number of second values of all Boolean events of the plurality of Boolean event series within a first time window, determining, by the computing system, a second total number of second values of all Boolean events of the plurality of Boolean event series within a second time window; determining, by the computing system, a first total number of Boolean events of the plurality of Boolean event series within the first time window; determining, by the computing system, a second total number of Boolean events of the plurality of Boolean event series with the second time window, wherein the first time window is less than the time period, wherein the second time window is less than the time period; determining, by the computing system, a population second value rate for all Boolean events of the plurality of Boolean event series within the time period; determining, by the computing system, a first p-value for the first time window based on the first total number of second values, the first total number of Boolean events, and the population second value rate; determining, by the computing system, a second p-value for the second time window based on the second total number of second values, the second total number of Boolean events, and the population second value rate; determining, by the computing system, whether the first time window includes a systemic fault based on the first p-value being less than or equal to a threshold p-value; and determining, by the computing system, whether the second time window includes a systemic fault based on the second p-value being less than or equal to a threshold p-value.

Example 9: A computing system comprising: a communication unit configured to obtain a plurality of Boolean events; and one or more processors implemented in circuitry and in communication with the communication unit, the one or more processors configured to: obtain a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determine an actual number of switch events in the series of Boolean events, wherein each switch event in the series of Boolean events is a change from the first value to the second value or the second value to the first value; determine an expected number of switch events for the series of Boolean events; determine, based on the expected number of switch events and the total number of Boolean events in the series of Boolean events, a confidence interval for the expected number of switch events; determine a score based on the actual number of switch events, the expected number of switch events, and the confidence interval, wherein the score is indicative of a fault in a process generating the series of Boolean events; and generate an alert based on the score being greater than or equal to a threshold score.

Example 10: The computing system of example 9, wherein determining the expected number of switch events is based on a total number of Boolean events in the series of Boolean events being the first value and the total number of Boolean events.

Example 11: The computing system of example 9 or example 10, wherein the score is determined based on an Agresti-Coull method.

Example 12: The computing system of any one of examples 9 through 11, wherein the series of Boolean events are ordered in time.

Example 13: The computing system of any one of examples 9 through 12, wherein the service is a healthcare service, wherein the service is provided by a healthcare provider.

Example 14: The computing system of any one of examples 9 through 13, wherein the series of Boolean events is a first series of Boolean events, wherein the total number of Boolean events is a first total number of Boolean events, wherein the service is a first service, wherein the actual number of switch events is a first actual number of switch events, wherein the expected number of switch events is a first expected number of switch events, wherein the confidence interval is a first confidence interval, wherein the score is a first score, the one or more processors further configured to: obtain a second series of Boolean events, wherein: a second total number of Boolean events in the second series of Boolean events is greater than two, and each respective Boolean event in the second series of Boolean events has either the first value or the second value, the first value indicating that reimbursement for a respective overpayment of a second service was timely received, the second value indicating that reimbursement for the respective overpayment of the second service was not timely received; determine a second actual number of switch events in the second series of Boolean events, wherein each switch event in the second series of Boolean events is a change from the first value to the second value or the second value to the first value; determine a second expected number of switch events for the second series of Boolean events; determine based on the second expected number of switch events and the second total number of Boolean events in the series of Boolean events, a second confidence interval for a Bernoulli process; determine a second score based on the second actual number of switch events, the second expected number of switch events, and the second confidence interval, wherein the second score characterizes how well the second series of Boolean events is modeled by the Bernoulli process; and generate an alert based on the second score being greater than or equal to a threshold score.

Example 15: The computing system of example 14, wherein the one or more processors further configured to determine a metric indicative of a systemic fault affecting a plurality of Boolean event series within a time period.

Example 16: The computing system of example 15, wherein the one or more processors further configured to: determine a first total number of second values of all Boolean events of the plurality of Boolean event series within a first time window, determine a second total number of second values of all Boolean events of the plurality of Boolean event series within a second time window; determine a first total number of Boolean events of the plurality of Boolean event series within the first time window; determine a second total number of Boolean events of the plurality of Boolean event series with the second time window, wherein the first time window is less than the time period, wherein the second time window is less than the time period; determine a population second value rate for all Boolean events of the plurality of Boolean event series within the time period; determine a first p-value for the first time window based on the first total number of second values, the first total number of Boolean events, and the population second value rate; determine a second p-value for the second time window based on the second total number of second values, the second total number of Boolean events, and the population second value rate; determine whether the first time window includes a systemic fault based on the first p-value being less than or equal to a threshold p-value; and determine whether the second time window includes a systemic fault based on the second p-value being less than or equal to a threshold p-value.

Example 17: A non-transitory computer-readable medium having instructions stored thereon that, when executed, cause one or more processors to: obtain a series of Boolean events, wherein: a total number of Boolean events in the series of Boolean events is greater than two, and each respective Boolean event in the series of Boolean events has either a first value or a second value, the first value indicating that reimbursement for a respective overpayment of a service was timely received, the second value indicating that reimbursement for the respective overpayment of the service was not timely received; determine an actual number of switch events in the series of Boolean events, wherein each switch event in the series of Boolean events is a change from the first value to the second value or the second value to the first value; determine an expected number of switch events for the series of Boolean events; determine, based on the expected number of switch events and the total number of Boolean events in the series of Boolean events, a confidence interval for the expected number of switch events; determine a score based on the actual number of switch events, the expected number of switch events, and the confidence interval, wherein the score is indicative of a fault in a process generating the series of Boolean events; and generate an alert based on the score being greater than or equal to a threshold score.

Example 18: The non-transitory computer-readable medium of example 17, wherein determining the expected number of switch events is based on a total number of Boolean events in the series of Boolean events being the first value and the total number of Boolean events.

Example 19: The non-transitory computer-readable medium of example 17 or example 18, wherein the score is determined based on an Agresti-Coull method.

Example 20: The non-transitory computer-readable medium of any one of examples 17 through 19, wherein the service is a healthcare service, wherein the service is provided by a healthcare provider.

For processes, apparatuses, and other examples or illustrations described herein, including in any flowcharts or flow diagrams, certain operations, acts, steps, or events included in any of the techniques described herein can be performed in a different sequence, may be added, merged, or left out altogether (e.g., not all described acts or events are necessary for the practice of the techniques). Moreover, in certain examples, operations, acts, steps, or events may be performed concurrently, e.g., through multi-threaded processing, interrupt processing, or multiple processors, rather than sequentially. Further certain operations, acts, steps, or events may be performed automatically even if not specifically identified as being performed automatically. Also, certain operations, acts, steps, or events described as being performed automatically may be alternatively not performed automatically, but rather, such operations, acts, steps, or events may be, in some examples, performed in response to input or another event.

Further, certain operations, techniques, features, and/or functions may be described herein as being performed by specific components, devices, and/or modules. In other examples, such operations, techniques, features, and/or functions may be performed by different components, devices, or modules. Accordingly, some operations, techniques, features, and/or functions that may be described herein as being attributed to one or more components, devices, or modules may, in other examples, be attributed to other components, devices, and/or modules, even if not specifically described herein in such a manner.

In one or more examples, the functions described may be implemented in hardware, software, firmware, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers, processing circuitry, or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM, or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Instructions may be executed by processing circuitry (e.g., one or more processors, such as one or more DSPs, general purpose microprocessors, ASICs, FPGAs, or other equivalent integrated or discrete logic circuitry), as well as any combination of such components. Accordingly, the term “processor” or “processing circuitry” as used herein, may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device or wireless handset, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A method comprising:
obtaining, by a computing system, a series of Boolean events, wherein:
a total number of Boolean events in the series of Boolean events is greater than two, and
each respective Boolean event in the series of Boolean events has either a first value or a second value, the first value indicating that reimbursement for a respective overpayment of a service was timely received, the second value indicating that reimbursement for the respective overpayment of the service was not timely received;
determining, by the computing system, an actual number of switch events in the series of Boolean events, wherein each switch event in the series of Boolean events is a change from the first value to the second value or the second value to the first value;
determining, by the computing system, an expected number of switch events for the series of Boolean events;
determining, by the computing system, based on the expected number of switch events and the total number of Boolean events in the series of Boolean events, a confidence interval for the expected number of switch events;
determining, by the computing system, a score based on the actual number of switch events, the expected number of switch events, and the confidence interval, wherein the score is indicative of a fault in a process generating the series of Boolean events; and
generating, by the computing system, an alert based on the score being greater than or equal to a threshold score.

2. The method of claim 1, wherein determining the expected number of switch events is based on a total number of Boolean events in the series of Boolean events being the first value and the total number of Boolean events.

3. The method of claim 1, wherein the score is determined based on an Agresti-Coull method.

4. The method of claim 1, wherein the series of Boolean events are ordered in time.

5. The method of claim 1, wherein the service is a healthcare service, wherein the service is provided by a healthcare service provider.

6. The method of claim 1, wherein the series of Boolean events is a first series of Boolean events, wherein the total number of Boolean events is a first total number of Boolean events, wherein service is a first service, wherein the actual number of switch events is a first actual number of switch events, wherein the expected number of switch events is a first expected number of switch events, wherein the confidence interval is a first confidence interval, wherein the score is a first score, the method further comprising:
obtaining, by a computing system, a second series of Boolean events, wherein:
a second total number of Boolean events in the second series of Boolean events is greater than two, and
each respective Boolean event in the second series of Boolean events has either the first value or the second value, the first value indicating that reimbursement for a respective overpayment of a second service was timely received, the second value indicating that reimbursement for the respective overpayment of the second service was not timely received;
determining, by the computing system, a second actual number of switch events in the second series of Boolean events, wherein each switch event in the second series of Boolean events is a change from the first value to the second value or the second value to the first value;
determining, by the computing system, a second expected number of switch events for the second series of Boolean events;
determining, by the computing system, based on the second expected number of switch events and the second total number of Boolean events in the series of Boolean events, a second confidence interval for a Bernoulli process;
determining, by the computing system, a second score based on the second actual number of switch events, the second expected number of switch events, and the second confidence interval, wherein the second score characterizes how well the second series of Boolean events is modeled by the Bernoulli process; and
generating, by the computing system, an alert based on the second score being greater than or equal to a threshold score.

7. The method of claim 6, further comprising:
determining, by the computing system, a metric indicative of a systemic fault affecting a plurality of Boolean event series within a time period.

8. The method of claim 7, wherein determining the metric indicative of a systemic fault comprises:
determining, by the computing system, a first total number of second values of all Boolean events of the plurality of Boolean event series within a first time window,
determining, by the computing system, a second total number of second values of all Boolean events of the plurality of Boolean event series within a second time window;
determining, by the computing system, a first total number of Boolean events of the plurality of Boolean event series within the first time window;
determining, by the computing system, a second total number of Boolean events of the plurality of Boolean event series with the second time window, wherein the first time window is less than the time period, wherein the second time window is less than the time period;
determining, by the computing system, a population second value rate for all Boolean events of the plurality of Boolean event series within the time period;
determining, by the computing system, a first p-value for the first time window based on the first total number of second values, the first total number of Boolean events, and the population second value rate;
determining, by the computing system, a second p-value for the second time window based on the second total number of second values, the second total number of Boolean events, and the population second value rate;
determining, by the computing system, whether the first time window includes a systemic fault based on the first p-value being less than or equal to a threshold p-value; and
determining, by the computing system, whether the second time window includes a systemic fault based on the second p-value being less than or equal to a threshold p-value.

9. A computing system comprising:
a communication unit configured to obtain a plurality of Boolean events; and
one or more processors implemented in circuitry and in communication with the communication unit, the one or more processors configured to:
obtain a series of Boolean events, wherein:
a total number of Boolean events in the series of Boolean events is greater than two, and
each respective Boolean event in the series of Boolean events has either a first value or a second value, the first value indicating that reimbursement for a respective overpayment of a service was timely received, the second value indicating that reimbursement for the respective overpayment of the service was not timely received;
determine an actual number of switch events in the series of Boolean events, wherein each switch event in the series of Boolean events is a change from the first value to the second value or the second value to the first value;
determine an expected number of switch events for the series of Boolean events;
determine, based on the expected number of switch events and the total number of Boolean events in the series of Boolean events, a confidence interval for the expected number of switch events;
determine a score based on the actual number of switch events, the expected number of switch events, and the confidence interval, wherein the score is indicative of a fault in a process generating the series of Boolean events; and
generate an alert based on the score being greater than or equal to a threshold score.

10. The computing system of claim 9, wherein determining the expected number of switch events is based on a total number of Boolean events in the series of Boolean events being the first value and the total number of Boolean events.

11. The computing system of claim 9, wherein the score is determined based on an Agresti-Coull method.

12. The computing system of claim 9, wherein the series of Boolean events are ordered in time.

13. The computing system of claim 9, wherein the service is a healthcare service, wherein the service is provided by a healthcare provider.

14. The computing system of claim 9, wherein the series of Boolean events is a first series of Boolean events, wherein the total number of Boolean events is a first total number of Boolean events, wherein the service is a first service, wherein the actual number of switch events is a first actual number of switch events, wherein the expected number of switch events is a first expected number of switch events, wherein the confidence interval is a first confidence interval, wherein the score is a first score, the one or more processors further configured to:
obtain a second series of Boolean events, wherein:
a second total number of Boolean events in the second series of Boolean events is greater than two, and
each respective Boolean event in the second series of Boolean events has either the first value or the second value, the first value indicating that reimbursement for a respective overpayment of a second service was timely received, the second value indicating that reimbursement for the respective overpayment of the second service was not timely received;
determine a second actual number of switch events in the second series of Boolean events, wherein each switch event in the second series of Boolean events is a change from the first value to the second value or the second value to the first value;
determine a second expected number of switch events for the second series of Boolean events;
determine based on the second expected number of switch events and the second total number of Boolean events in the series of Boolean events, a second confidence interval for a Bernoulli process;
determine a second score based on the second actual number of switch events, the second expected number of switch events, and the second confidence interval, wherein the second score characterizes how well the second series of Boolean events is modeled by the Bernoulli process; and
generate an alert based on the second score being greater than or equal to a threshold score.

15. The computing system of claim 14, wherein the one or more processors further configured to determine a metric indicative of a systemic fault affecting a plurality of Boolean event series within a time period.

16. The computing system of claim 15, wherein the one or more processors further configured to:
determine a first total number of second values of all Boolean events of the plurality of Boolean event series within a first time window,
determine a second total number of second values of all Boolean events of the plurality of Boolean event series within a second time window;
determine a first total number of Boolean events of the plurality of Boolean event series within the first time window;
determine a second total number of Boolean events of the plurality of Boolean event series with the second time window, wherein the first time window is less than the time period, wherein the second time window is less than the time period;
determine a population second value rate for all Boolean events of the plurality of Boolean event series within the time period;
determine a first p-value for the first time window based on the first total number of second values, the first total number of Boolean events, and the population second value rate;
determine a second p-value for the second time window based on the second total number of second values, the second total number of Boolean events, and the population second value rate;
determine whether the first time window includes a systemic fault based on the first p-value being less than or equal to a threshold p-value; and
determine whether the second time window includes a systemic fault based on the second p-value being less than or equal to a threshold p-value.

17. A non-transitory computer-readable medium having instructions stored thereon that, when executed, cause one or more processors to:
obtain a series of Boolean events, wherein:
a total number of Boolean events in the series of Boolean events is greater than two, and
each respective Boolean event in the series of Boolean events has either a first value or a second value, the first value indicating that reimbursement for a respective overpayment of a service was timely received, the second value indicating that reimbursement for the respective overpayment of the service was not timely received;
determine an actual number of switch events in the series of Boolean events, wherein each switch event in the series of Boolean events is a change from the first value to the second value or the second value to the first value;
determine an expected number of switch events for the series of Boolean events;
determine, based on the expected number of switch events and the total number of Boolean events in the series of Boolean events, a confidence interval for the expected number of switch events;
determine a score based on the actual number of switch events, the expected number of switch events, and the confidence interval, wherein the score is indicative of a fault in a process generating the series of Boolean events; and
generate an alert based on the score being greater than or equal to a threshold score.

18. The non-transitory computer-readable medium of claim 17, wherein determining the expected number of switch events is based on a total number of Boolean events in the series of Boolean events being the first value and the total number of Boolean events.

19. The non-transitory computer-readable medium of claim 17, wherein the score is determined based on an Agresti-Coull method.

20. The non-transitory computer-readable medium of claim 17, wherein the service is a healthcare service, wherein the service is provided by a healthcare provider.
